(19) United States
(12) Patent Application Publication
(10) Pub. No.: US 2023/0016752 A1
(43) Pub. Date: Jan. 19, 2023

(54) GASKET WITH EXTERNAL SEALING FEATURES FOR INTRODUCER SHEATH

(71) Applicant: Boston Scientific Scimed, Inc., Maple Grove, MN (US)

(72) Inventors: Arthur J. FOSTER, Blaine, MN (US); Kathleen E. THYKEN, Shoreview, MN (US); Steven E. SCHULTZ, West Lakeland, MN (US); Brian P. EDISON, Otsego, MN (US)

(21) Appl. No.: 17/867,256

(22) Filed: Jul. 18, 2022

Related U.S. Application Data
(60) Provisional application No. 63/223,444, filed on Jul. 19, 2021.

(51) Int. Cl. A61M 39/06

(52) U.S. Cl. CPC A61M 39/06; A61M 2039/0626; A61M 2039/0258

(57) ABSTRACT

A hemostasis seal member for a medical device, the seal member comprises a cylindrical main body portion having a proximal face, a distal face, and a peripheral surface face extending between the proximal and distal faces, the main body portion having a first diameter, and a radial sealing feature extending radially inward or outward from the peripheral surface and configured to sealingly engage an inner cylindrical surface of a hemostasis valve housing. The radial sealing feature comprises a radial projection extending radially outward from the peripheral surface, or a concave recess extending radially inward from the peripheral surface.

### CROSS-REFERENCE TO RELATED APPLICATION

This application claims priority to U.S. Provisional Patent Application No. 63/223,444, filed on Jul. 19, 2021, in the United States Patent and Trademark Office. The disclosure of which is incorporated herein by reference in its entirety.

### TECHNICAL FIELD

The present disclosure relates to medical introducer sheaths for introducing medical devices into the body. In particular, the present disclosure relates to introducer sheaths and associated hemostasis valves.

### BACKGROUND

Introducer sheaths are important instruments for use in medical diagnostic and therapeutic procedures. In particular, introducer sheaths can be used by a clinician to access target areas of the patient's anatomy, e.g., the patient's cardiovascular system, and also allow for diagnostic and/or therapeutic payloads (such as ablation catheters) to be inserted into these target areas. Introducer sheaths often include hemostasis valves that permit introduction of the desired payloads but at the same time inhibit undesired fluid loss from the introducer sheath.

### SUMMARY

In Example 1, a hemostasis valve assembly for an introducer sheath, the hemostasis valve assembly comprising a housing and a seal member. The housing has a proximal opening configured to permit introduction of an instrument therethrough, and defines an inner surface. The seal member is positioned distal to the proximal opening, and comprises a main body portion and a radial sealing feature. The main body portion has a proximal face, a distal face, and a peripheral surface extending between the proximal and distal faces, and has a first diameter defined by the peripheral surface. The radial sealing feature extends radially inward or outward from the peripheral surface and is configured to sealingly engage the inner surface of the housing.

In Example 2, the hemostasis valve assembly of Example 1, wherein the radial sealing feature comprises a radial projection extending radially outward from and circumferentially about the peripheral surface of the main body portion, a radial extremity of the radial projection defining a second diameter greater than the first diameter, wherein the radial projection sealingly engages the inner surface of the housing.

In Example 3, the hemostasis valve assembly of Example 2, wherein the radial projection has a semi-circular cross-sectional profile.

In Example 4, the hemostasis valve assembly of Example 2, wherein the radial projection has first and second side faces extending radially from the peripheral surface, and a perimeter face extending from the first side face to the second side face.

In Example 5, the hemostasis valve assembly of Example 4, wherein the perimeter face has a generally semi-circular profile.

In Example 6, the hemostasis valve assembly of Example 2, wherein the radial projection has an outer surface that extends from the peripheral surface at an oblique angle relative to an axis extending through the proximal and distal faces.

In Example 7, the hemostasis valve assembly of Example 1, wherein the inner surface of the housing includes an annular projection, and wherein the radial sealing feature comprises a concave recess extending radially inward from the peripheral surface, the concave recess having a cross-sectional profile configured to receive the annular projection of the housing in sealing engagement therewith.

In Example 8, the hemostasis valve assembly of Example 1, wherein the radial sealing feature comprises a plurality of spaced radial projections each extending radially outward of and circumferentially about the peripheral surface of the main body, wherein each radial projection is in sealing engagement with the inner surface of the housing.

In Example 9, the hemostasis valve assembly of Example 8, wherein each radial projection has a semi-circular cross-sectional profile.

In Example 10, the hemostasis valve assembly of Example 8, wherein each radial projection has an outer surface that extends from the peripheral surface at an oblique angle relative to an axis extending through the proximal and distal faces.

In Example 11, the hemostasis valve assembly of Example 8, wherein each radial projection has first and second side faces extending radially from the peripheral surface, and a perimeter face extending from the first side face to the second side face.

In Example 12, the hemostasis valve assembly of any of Examples 1-11, wherein the hemostasis seal further comprises an annular side recess in the proximal face or the distal face of the main body, and wherein the housing includes an annular side projection received within and in sealing engagement with the annular side recess.

In Example 13, the hemostasis valve assembly of any of Examples 1-11, wherein the hemostasis seal further comprises an annular side projection extending from the proximal face or the distal face of the main body, and wherein the housing includes an annular side recess that receives the annular side projection in sealing engagement therewith.

In Example 14, the hemostasis valve assembly of any of Examples 1-13, wherein the hemostasis valve further comprises a perforation or slit extending from the proximal face to the distal face to permit passage of a medical device therethrough.

In Example 15, the introducer sheath assembly comprising a tubular sheath member having a proximal end portion including a proximal end, a distal end portion terminating in a distal tip, and an internal sheath lumen extending through the proximal and distal end portions of the sheath member, and the hemostasis valve assembly of any of Examples 1-14 coupled to the proximal end of the sheath member.

In Example 16, the introducer sheath assembly comprising a tubular sheath member and a hemostasis valve assembly. The tubular sheath member has a proximal end portion including a proximal end, a distal end portion terminating in a distal tip, and an internal sheath lumen extending through the proximal and distal end portions of the sheath member. The hemostasis valve assembly is coupled to the proximal end of the sheath member, and comprises a housing and a seal member. The housing is configured to permit introduction of an instrument therethrough, and defines an inner surface. The seal member is configured to inhibit fluid leakage from the hemostasis valve assembly, and comprises a main body portion and a radial sealing feature. The main body portion has a proximal face, a distal face, and a peripheral surface extending between the proximal and distal faces, and has a first diameter defined by the peripheral surface. The radial sealing feature extends radially inward or outward from the peripheral surface and is configured to sealingly engage the inner surface of the housing.

In Example 17, the introducer sheath assembly of Example 16, wherein the radial sealing feature comprises a radial projection extending radially outward from and circumferentially about the peripheral surface of the main body portion, a radial extremity of the radial projection defining a second diameter greater than the first diameter, wherein the radial projection sealingly engages the inner surface of the housing.

In Example 18, the introducer sheath assembly of Example 17, wherein the radial projection has a semi-circular cross-sectional profile.

In Example 19, the introducer sheath assembly of Example 17, wherein the radial projection has first and second side faces extending radially from the peripheral surface, and a perimeter face extending from the first side face to the second side face.

In Example 20, the introducer sheath assembly of Example 17, wherein the radial projection has an outer surface that extends from the peripheral surface at an oblique angle relative to an axis extending through the proximal and distal faces.

In Example 21, the introducer sheath assembly of Example 16, wherein the radial sealing feature comprises a plurality of spaced radial projections each extending radially outward of and circumferentially about the peripheral surface of the main body, wherein each radial projection is in sealing engagement with the inner surface of the housing.

In Example 22, the introducer sheath assembly of Example 21, wherein each radial projection has a semi-circular cross-sectional profile.

In Example 23, the introducer sheath assembly of Example 21, wherein each radial projection has first and second side faces extending radially from the peripheral surface, and a perimeter face extending from the first side face to the second side face.

In Example 24, the introducer sheath assembly of Example 16, wherein the inner surface of the housing includes an annular projection, and wherein the radial sealing feature comprises a concave recess extending radially inward from the peripheral surface, the concave recess having a cross-sectional profile configured to receive the annular projection of the housing in sealing engagement therewith.

In Example 25, the hemostasis valve assembly for an introducer sheath having a sheath lumen, the hemostasis valve assembly comprising a housing having a proximal opening configured to be generally aligned with the sheath lumen to permit introduction of an instrument therethrough, the housing defining an inner surface, and a seal member positioned distal to the proximal opening to inhibit fluid leakage through the proximal opening. The seal member comprises a main body portion having a proximal face, a distal face, and a peripheral surface extending between the proximal and distal faces, the main body portion having a first diameter defined by the peripheral surface, and a radial sealing feature extending radially inward or outward from the peripheral surface and configured to sealingly engage the inner surface of the housing.

In Example 26, the hemostasis valve assembly of Example 25, wherein the radial sealing feature comprises a radial projection extending radially outward from and circumferentially about the peripheral surface of the main body portion, a radial extremity of the radial projection defining a second diameter greater than the first diameter, wherein the radial projection sealingly engages the inner surface of the housing.

In Example 27, the hemostasis valve assembly of Example 26, wherein the radial projection has a semi-circular cross-sectional profile.

In Example 28, the hemostasis valve assembly of Example 26, wherein the radial projection has first and second side faces extending radially from the peripheral surface, and a perimeter face extending from the first side face to the second side face.

In Example 29, the hemostasis valve assembly of Example 25, wherein the inner surface of the housing includes an annular projection, and wherein the radial sealing feature comprises a concave recess extending radially inward from the peripheral surface, the concave recess having a cross-sectional profile configured to receive the annular projection of the housing in sealing engagement therewith.

In Example 30, the hemostasis seal member for a medical device. The seal member comprises a generally cylindrical main body portion having a proximal face, a distal face, and a peripheral surface extending between the proximal and distal faces, the main body portion having a first diameter, and a radial sealing feature extending radially inward or outward from the peripheral surface and configured to sealingly engage an inner surface of a hemostasis valve housing.

In Example 31, the hemostasis seal member of Example 30, wherein the radial sealing feature comprises a radial projection extending radially outward from and circumferentially about the peripheral surface of the main body portion, a radial extremity of the radial projection defining a second diameter greater than the first diameter.

In Example 32, the hemostasis seal member of Example 31, wherein the radial projection has a semi-circular cross-sectional profile.

In Example 33, the hemostasis seal member of Example 31, wherein the radial projection has first and second side faces extending radially from the peripheral surface, and a perimeter face extending from the first side face to the second side face.

In Example 34, the hemostasis seal member of Example 30, wherein the radial sealing feature comprises a plurality of spaced radial projections each extending radially outward of and circumferentially about the peripheral surface of the main body.

In Example 35, the introducer sheath assembly of Example 30, wherein the radial sealing feature comprises a concave recess extending radially inward from the peripheral surface, the concave recess having a cross-sectional profile configured to receive an annular projection of the housing in sealing engagement therewith.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1A is an isometric view of an exemplary introducer sheath assembly according to one embodiment of the present disclosure.

FIG. 1B is a plan view of the introducer sheath assembly of FIG. 1A illustrating the deflectability of the sheath according to one embodiment of the present disclosure.

FIG. 2 is a cross-sectional elevation view of the hemostasis valve assembly of the introducer sheath assembly of FIG. 1A according to one embodiment of the present disclosure.

FIGS. 3A-3B are isometric and cross-sectional views, respectively, of a hemostasis seal member for the hemostasis valve assembly of FIGS. 2 and 3, according to one embodiment of the present disclosure.

FIGS. 4A-4B are isometric and cross-sectional views, respectively, of an alternative hemostasis seal member according to alternative embodiments of the present disclosure.

FIGS. 5-10 are cross-sectional views of additional hemostasis seal members according to alternative embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

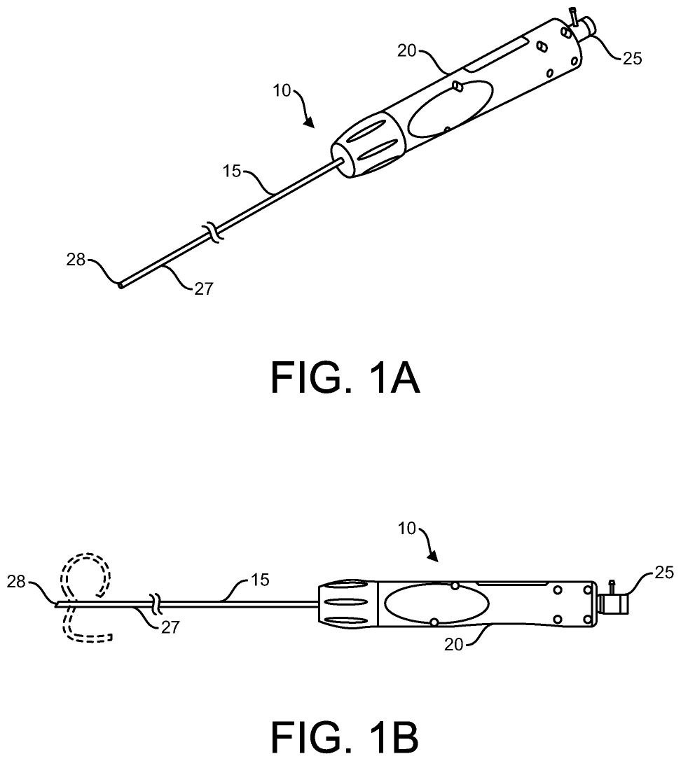

FIG. 1A is an isometric view of an exemplary introducer sheath assembly 10 according to one embodiment of the present invention. The introducer sheath assembly 10 is configured for use in a broad range of medical procedures requiring the introduction of a payload into the body. For example, in various embodiments, the introducer sheath assembly 10 can be used to introduce therapeutic and/or diagnostic electrophysiology catheters, e.g., ablation and/or mapping catheters, into a heart chamber. In other embodiments, the introducer sheath assembly 10 can be used to deliver implantable devices, e.g., pacing leads, to a heart chamber or to a coronary vein. In short, the present invention is not limited to any particular clinical use.

As shown in FIG. 1A, the introducer sheath assembly 10 includes a sheath member 15, a handle assembly 20 and a hemostasis valve assembly 25. In the illustrated embodiment, the handle assembly 20 is coupled to the sheath member 15 distal to the hemostasis valve assembly 25, which occupies the proximal-most position on the introducer sheath assembly 10. As will be explained in further detail below, the sheath member 15 includes a lumen extending longitudinally therethrough sized to allow introduction of a payload into the introducer sheath assembly 10 and out the distal end of the sheath member 15. Additionally, in various embodiments, the sheath member 15 includes a deflectable distal end portion 27 that can assume a variety of different shapes for navigating the patient anatomy, and as shown in FIG. 1A, terminates in a distal tip 28.

In general, the handle assembly 20 is configured to allow a clinician to manipulate and control the introducer sheath assembly 10, and in particular, includes a mechanism for altering the shape of the distal end portion 27 of the sheath member 15 to assist in navigating the patient anatomy, as explained in further detail below. The hemostasis valve assembly 25 is configured to allow introduction of a payload into the lumen of the sheath member 15 while at the same time inhibiting unintended leakage of fluid from the sheath member 15 into the operating environment and/or aspiration of air into the sheath member 15 lumen.

In the illustrated embodiment, the introducer sheath assembly 10 is a deflectable or steerable introducer sheath, in that the shape or profile of at least the distal end portion 27 of the sheath member 15 can be altered, i.e., by manipulation of a steering mechanism in the handle assembly 20, to facilitate navigation of the patient anatomy. FIG. 1B is a plan view of the introducer sheath assembly 10 illustrating the deflectability of the sheath member 15. As indicated by the phantom lines in FIG. 1B, the distal end portion 27 can be deflected relative to its “straight” or undeflected state shown in FIG. 1A.

Deflection of the distal end portion 27 of the sheath member 15 can be effectuated by the inclusion of pull wires (not shown) extending within the sheath member 15 and fixedly connected to the sheath member 15 within the distal end portion 27 at one end, and to the handle assembly 20 at the other end. In various embodiments, these pull wires are each secured to the sheath member 15 near its distal tip 28 (e.g., by attaching the pull wires to an anchor member incorporated into the sheath member 15) such that the distal ends of the pull wires are fixed relative to the sheath member 15. Accordingly, by selectively applying tension to the respective pull wires (e.g., via the steering mechanism incorporated into the handle assembly 20), the distal end portion 27 is deflected. It is noted that the embodiments of the present invention are not in any way limited by the technique by which the pull wires (or other deflection means) are routed through the sheath member 15, secured to the distal end portion 27 thereof, or whether the sheath member 15 is even deflectable.

In addition, except as otherwise discussed herein, the particular construction of the sheath member 15 in the various embodiments is not critical, and thus will not be discussed in great detail. In short, any number of construction details can be utilized for the sheath member 15 within the scope of the embodiments of the present invention. For example, the sheath member can have a single or multiple layer polymer construction, and may or may not include one or more reinforcing elements (e.g., braids, coils, wires, etc.) to enhance the mechanical characteristics, e.g., stiffness, torquability and the like, of the sheath member 15. In one embodiment, the sheath member 15 includes an outer layer of a biocompatible polymer such as a polyether block amide, an inner layer of a lubricious polymeric material such as polytetrafluoroethylene (PTFE), ethylene tetrafluoroethylene (ETFE), silicone, and the like, with a stainless steel braid and/or stainless steel wire coil embedded between the outer and inner polymer layers. In various embodiments, the durometer of the polymeric materials making up the outer and/or inner layers may be varied along the length of the sheath member 15 to further tailor the mechanical characteristics to the needs of the end user. Again, unless specifically discussed herein, the construction of the sheath member 15 is in no way limited to any particular construction.

It is emphasized that the hemostasis valve assembly 25 of the present disclosure, and the claims appended hereto, are not limited to use on a deflectable or steerable sheath. To the contrary, the hemostasis valve assembly 25 can be readily and advantageously used on other types of introducer sheaths and catheters, for example, fixed-shape introducer sheaths and guide catheters.

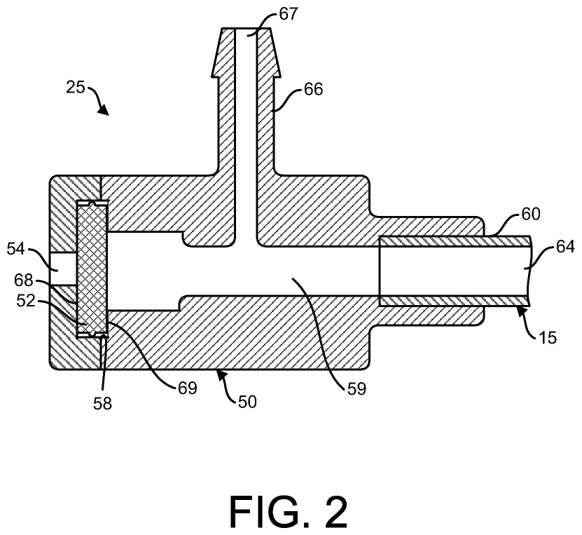

FIG. 2 is a cross-sectional elevation view of the hemostasis valve assembly 25 and proximal end of the sheath member 15 according to one embodiment of the present disclosure. As shown in FIG. 2, the hemostasis valve assembly 25 has, in relevant part, a housing 50 and a seal member 52 disposed therewithin. As further shown, the housing 50 has a proximal opening 54 and an inner surface 58, which in embodiments may be a cylindrical surface, which defines an internal channel 59. As can be further seen in FIG. 2, the housing is secured to a proximal end portion 60 of the introducer sheath member 15, which includes a sheath lumen 64 that is in fluid communication with the internal channel 59. In the illustrated embodiment, the housing 50 further includes a side port member 66 defining a side port lumen 68 that is also in fluid communication with the internal channel 59 and, consequently, the sheath lumen 64. The side port member 66 can be connected to a syringe or other device for flushing the sheath lumen 64 or introduction of diagnostic or therapeutic fluids depending on the particular clinical needs. In general, the specific design of the housing 50 shown in FIG. 2 is not intended to be limiting, and any number of housing 50 configurations can be utilized within the scope of the present disclosure.

As can be seen in FIG. 2, the proximal opening 54 is generally aligned with the sheath lumen 64 to allow for introduction of a medical device, e.g., a delivery tool (such as a guidewire or stylet), a diagnostic or therapeutic device (such as a mapping catheter, an ablation catheter, and the like), or other medical device into a body cavity through the sheath lumen 64, depending on the particular medical procedure to be performed. As further shown, the seal member 52 is positioned distal to the proximal opening 54 to inhibit fluid leakage through the proximal opening 54. In the illustrated embodiment, the seal member 52 is firmly secured within the housing 50 by radial shoulders 68, 69 defined by the housing 50 geometry, although in embodiments other structural designs may be employed. Additionally, as will be explained in greater detail elsewhere herein, the seal member 52 includes novel radial sealing features that are configured to sealingly engage the inner surface 58 of the housing 50 to further enhance the integrity of the seal formed by the cooperation of the seal member 52 and the housing 50.

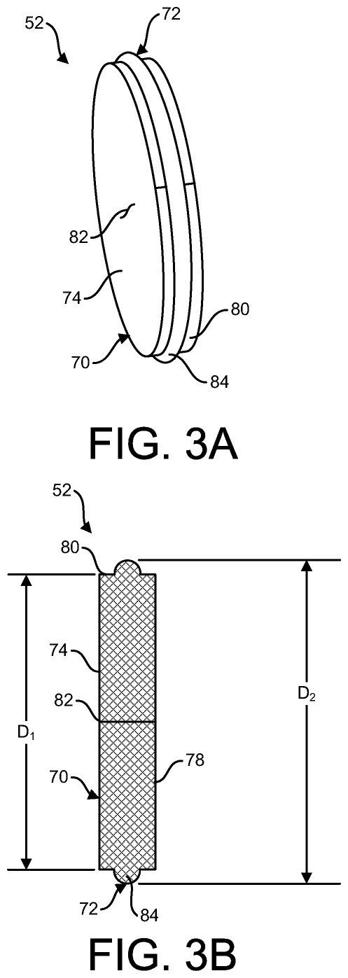

FIGS. 3A and 3B are isometric and cross-sectional elevation views, respectively, of the seal member 52 according to one embodiment of the present disclosure. As can be seen in FIGS. 3A and 3B collectively, the seal member 52 includes a main body 70 and a radial sealing feature 72. In the illustrated embodiment, the main body 70 is cylindrical so as to complement a cylindrical inner surface 58 of the housing 50. It will be appreciated, however, that in embodiments where the inner surface 58 of the housing 50 has a different internal geometry, e.g., polygonal, the geometry of the main body 70 can be adapted accordingly.

As further shown, the main body 70 includes a proximal face 74, a distal face 78, and a peripheral surface 80 extending between the proximal and distal faces 74, 78. As shown, the peripheral surface 80 defines a main body diameter Additionally, in embodiments, the main body 70 includes an access feature 82 extending through the thickness thereof to facilitate insertion of a medical device therethrough. For illustration purposes, the access feature 82 is shown as a slit through the main body 70 from the proximal face 74 to the distal face 78, although in other embodiments the access feature 82 can take on a different form (e.g., circular perforation, multiple intersecting slits, and the like). The particular configuration of the access feature 82 is not critical to the embodiments of the disclosure, and thus any suitable means can be employed to facilitate insertion of the medical device through the seal member 52.

In the illustrated embodiment, the radial sealing feature 72 is in the form of a radial projection 84 extending radially outward from and circumferentially about the peripheral surface 80 of the main body 70. As shown in FIG. 3B, a radial extremity of the radial projection 84 defines a diameter D2 greater than the diameter D1 of the main body 70. When positioned within the housing 50 for use, the radial projection 84 sealingly engages the inner surface 58 of the housing 50 (see FIG. 2). In the particular embodiment illustrated, the radial projection 84 has a generally semi-circular cross-sectional profile, although as explained elsewhere herein, in other embodiments the radial projection 84 can have different geometries.

In embodiments, the radial sealing feature 72 interacts with the inner surface 58 of the housing 50 to enhance the sealing capability of the combination of the housing 50 and seal member 52 to inhibit undesirable fluid leakage from the hemostasis valve assembly 25 through the proximal opening 54 of the housing 50. The radial sealing feature 72 functions to effectively lengthen potential leak paths between the housing 50 and the seal member 52 without appreciably increasing the overall compression of the seal member 52, which could have undesirable effects (e.g., increasing frictional resistance on the medical device being inserted through the access feature 82).

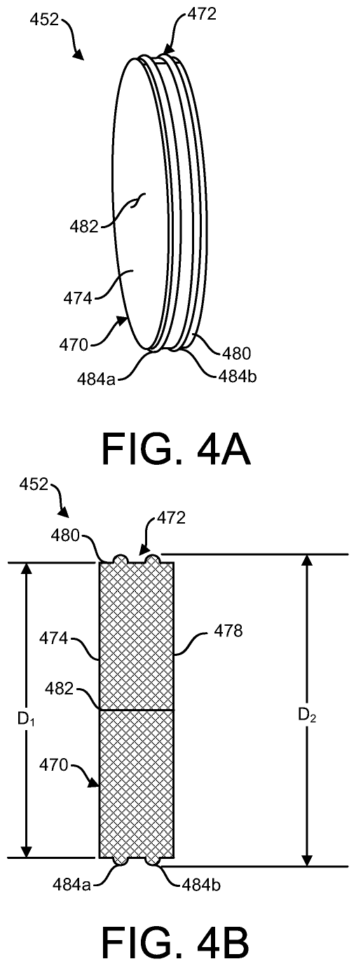

FIGS. 4A and 4B are isometric and cross-sectional elevation views, respectively, of an alternative seal member 452 for use in the hemostasis valve assembly 25 according to another embodiment of the present disclosure. As shown in FIGS. 4A and 4B, the seal member 452 is in many respects similar in construction to the seal member 52. Accordingly, the seal member 452 includes a main body 470 and a radial sealing feature 472. The main body 470 has a proximal face 474, a distal face 478 and a peripheral surface 480 between the proximal and distal faces 474, 478, as well as an access feature 482 extending through the thickness of the main body 470.

In the embodiment of FIGS. 4A and 4B, the radial sealing feature 472 comprises two radial projections 484a, 484b each having a generally semi-circular cross-sectional profile, similar to the single radial projection 84 of the seal member 52. The radial projections 484a and 484b otherwise operate and provide similar functionality and advantages to those described with respect to the radial projection 84 of the seal member 52. Furthermore, although two radial projections 484a, 484b are depicted in FIGS. 4A and 4B, in other embodiments, additional radial projections can be utilized to further enhance the sealing capability of the seal member 452.

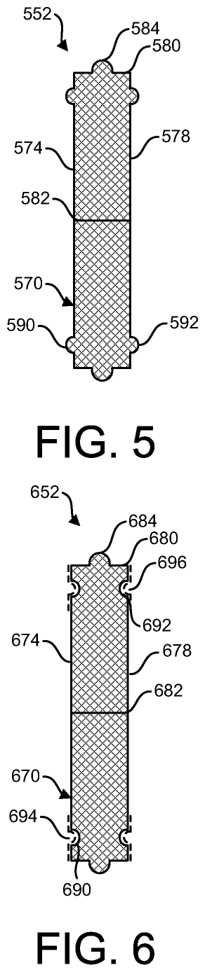

FIG. 5 is a cross-sectional elevation view of a seal member 552 according to another embodiment of the present disclosure. As shown in FIG. 5, the seal member 552 is in many respects similar in construction to the seal member 52 of FIGS. 3A-3B. Accordingly, the seal member 552 includes a main body 570 and a radial sealing feature in the form of a radial projection 584 having a generally semi-circular cross-sectional profile. The main body 570 has a proximal face 574, a distal face 578 and a peripheral surface 580 between the proximal and distal faces 574, 578, as well as an access feature 582 extending through the thickness of the main body 570.

In the embodiment of FIG. 5, the seal member 552 further includes annular side projections 590, 592 extending, respectively, from the proximal and distal faces 574, 578 near the peripheral surface 580. In embodiments, the side projections 590, 592 may interact with, e.g., be received within, corresponding annular side recesses (not depicted in FIG. 5) in the mating portions of the housing 50 (e.g., in the radial shoulders 68, 69, see FIG. 2) to further increase the length of potential leak paths between the seal member 552 and the corresponding housing 50. While the embodiment of FIG. 5 includes side projections 590, 592 on both the proximal and distal faces 574, 578, respectively, other embodiments may include only the side projection 590 or the side projection 592. Furthermore, in other embodiments, additional radial projections 584 (see, e.g., the embodiment of FIGS. 4A and 4B) may be utilized.

FIG. 6 is a cross-sectional elevation view of a seal member 652 according to another embodiment of the present disclosure. As shown in FIG. 6, the seal member 652 is in many respects similar in construction to the seal member 52 of FIGS. 3A-3B. Accordingly, the seal member 652 includes a main body 670 and a radial sealing feature in the form of a radial projection 684 having a generally semi-circular cross-sectional profile. The main body 670 has a proximal face 674, a distal face 678 and a peripheral surface 680 between the proximal and distal faces 674, 678, as well as an access feature 682 extending through the thickness of the main body 670.

In the embodiment of FIG. 6, the seal member 652 further includes annular side recesses 690, 692 in the proximal and distal faces 674, 678, respectively, near the peripheral surface 680. In embodiments, the side recesses 690, 692 may interact with, e.g., receive corresponding annular side projections 694, 696 (shown in phantom) in the mating portions of the housing 50 (e.g., in the radial shoulders 68, 69, see FIG. 2) to further increase the length of potential leak paths between the seal member 652 and the corresponding housing 50. While the embodiment of FIG. 6 includes side recesses 690, 692 on both the proximal and distal faces 674, 678, respectively, other embodiments may include only the side recess 690 or the side recess 692. Furthermore, in other embodiments, additional radial projections 684 (see, e.g., the embodiment of FIGS. 4A and 4B) may be utilized.

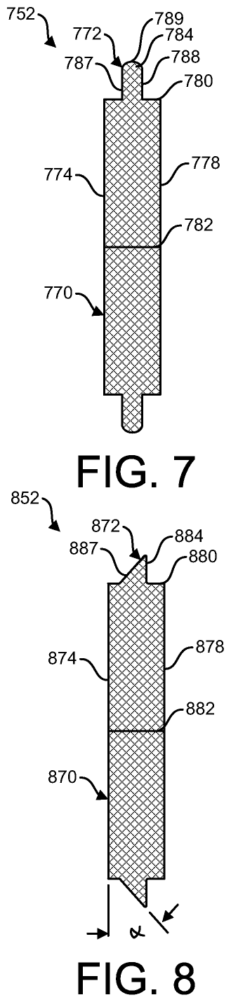

FIG. 7 is a cross-sectional elevation view of a seal member 752 according to another embodiment of the present disclosure. As shown FIG. 7, the seal member 752 is in many respects similar in construction to the seal member 52. Accordingly, the seal member 752 includes a main body 770 and a radial sealing feature 772. The main body 770 has a proximal face 774, a distal face 778 and a peripheral surface 780 between the proximal and distal faces 774, 778, as well as an access feature 782 extending through the thickness of the main body 770.

The seal member 752 differs from the seal member 52 of FIGS. 3A and 3B in the construction of the radial sealing feature 772. As shown in FIG. 7, the radial sealing feature 772 is in the form of an elongated radial projection 784 that has a proximal side surface 787, an opposite distal side surface 788, and an end surface 789 extending therebetween. The radial projection 784 functions similarly to the radial projection 84 of the seal member 52, while providing the additional advantage of being capable of deflecting laterally when inserted into the housing 50. As such, a relatively tight fit between the radial projection 784 and the inner surface of the housing 50 can be achieved without appreciably increasing radial compression on the seal member 784 as a whole, as discussed above. Although a single radial projection 784 is shown in FIG. 7, in other embodiments, additional radial projections 784 can be utilized within the scope of the present disclosure.

FIG. 8 is a cross-sectional elevation view of a seal member 852 according to another embodiment of the present disclosure. As shown FIG. 8, the seal member 852 is in many respects similar in construction to the seal member 52. Accordingly, the seal member 852 includes a main body 870 and a radial sealing feature 872. The main body 870 has a proximal face 874, a distal face 878 and a peripheral surface 880 between the proximal and distal faces 874, 878, as well as an access feature 882 extending through the thickness of the main body 870.

The seal member 852 differs from the seal member 52 of FIGS. 3A and 3B in the construction of the radial sealing feature 872. As shown in FIG. 8, the radial sealing feature 872 is in the form of a radial projection 884 that has a generally triangular cross-sectional profile and includes a side face 887 that extends from the peripheral surface 880 at an oblique angle a relative to a plane defined by the proximal and distal face 874.

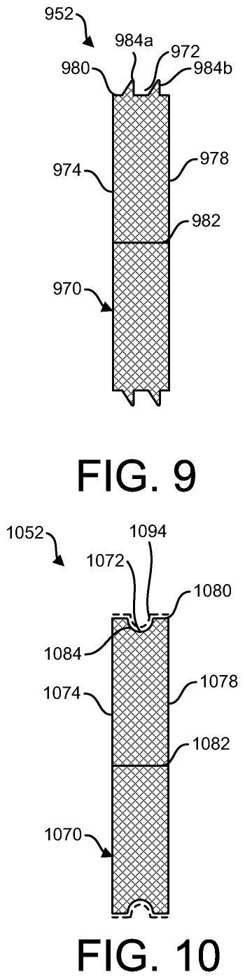

FIG. 9 is a cross-sectional elevation view of a seal member 952 according to another embodiment of the present disclosure. As shown FIG. 9, the seal member 952 is in many respects similar in construction to the seal member 852. Accordingly, the seal member 952 includes a main body 970 and a radial sealing feature 972. The main body 970 has a proximal face 974, a distal face 978 and a peripheral surface 980 between the proximal and distal faces 974, 978, as well as an access feature 982 extending through the thickness of the main body 970. In FIG. 9, the radial sealing feature 872 includes two radial projections 984a, 984b configured in the same way as the radial projection 884 of FIG. 8, i.e., each has a generally triangular cross-sectional profile and includes a side face that extends from the peripheral surface 980 at an oblique angle relative to a plane defined by the proximal and distal faces 974, 978. As will be appreciated, in additional embodiments, more than two triangular radial projections may be utilized within the scope of the present disclosure.

FIG. 10 is a cross-sectional elevation view of a seal member 1052 according to another embodiment of the present disclosure. As shown in FIG. 10, the seal member 1052 is in many respects similar in construction to the seal member 52 of FIGS. 3A-3B. Accordingly, the seal member 1052 includes a main body 1070 and a radial sealing feature 1072. The main body 1070 has a proximal face 1074, a distal face 1078 and a peripheral surface 1080 between the proximal and distal faces 1074, 1078, as well as an access feature 1082 extending through the thickness of the main body 1070.

In the embodiment of FIG. 10, the radial sealing feature 1072 is in the form of a radial recess 1084 extending radially inward of the peripheral surface 1080. As shown, the radial recess 1084 is configured to receive, in sealing engagement, a corresponding internal annular projection 1094 (shown in phantom in FIG. 10) formed in the adjacent inner surface 58 of the housing 50. Similar to the various radial projections depicted in the other illustrated embodiments, radial recess 1084 and mating annular projection 1094 of the housing 50 interact to enhance the sealing capability of the combination of the housing 50 and seal member 1052 to inhibit undesirable fluid leakage. In the illustrated embodiment, the radial recess 1084 has a generally semi-circular cross-sectional profile, although in other embodiment other profiles may be employed.

It will be appreciated that the radial sealing features of the various embodiments may be interchanged without departing from the cope of the present disclosure. In one non-limiting example, the side projections and/or side recesses shown in FIGS. 5 and 6 may optionally be employed in any of the embodiments depicted in FIGS. 4A-4B or FIGS. 7-10. Additionally, in embodiments utilizing more than one radial projection, the particular geometry of each radial projection can be varied, e.g., by combining semi-circular radial projections, elongated radial projections, or triangular radial projections. Furthermore, additional geometries for the radial projections and/or recesses may be utilized.

The housing 50 and the various seal members described herein can be constructed using any suitable biocompatible materials known today or later developed. In one embodiment, the seal members can be constructed of a wide range of medical grade silicones or comparable materials using conventional manufacturing techniques. In embodiments, the seal member materials can be treated, e.g., with lubricating materials or surface treatments, to enhance lubricity. In other embodiments, the seal member material may be augmented with additional functional materials, e.g., radiopaque additives to allow the seal member(s) or parts thereof to be visualized under fluoroscopy.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An introducer sheath assembly comprising:
a tubular sheath member having a proximal end portion including a proximal end, a distal end portion terminating in a distal tip, and an internal sheath lumen extending through the proximal and distal end portions of the sheath member;
a hemostasis valve assembly coupled to the proximal end of the sheath member, the hemostasis valve assembly comprising:
a housing having a proximal opening generally aligned with the sheath lumen to permit introduction of an instrument therethrough, the housing defining an inner surface; and
a seal member positioned distal to the proximal opening to inhibit fluid leakage through the proximal opening, the seal member comprising:
a main body portion having a proximal face, a distal face, and a peripheral surface extending between the proximal and distal faces, the main body portion having a first diameter defined by the peripheral surface; and
a radial sealing feature extending radially inward or outward from the peripheral surface and configured to sealingly engage the inner surface of the housing.

2. The introducer sheath assembly of claim 1, wherein the radial sealing feature comprises a radial projection extending radially outward from and circumferentially about the peripheral surface of the main body portion, a radial extremity of the radial projection defining a second diameter greater than the first diameter, wherein the radial projection sealingly engages the inner surface of the housing.

3. The introducer sheath assembly of claim 2, wherein the radial projection has a semi-circular cross-sectional profile.

4. The introducer sheath assembly of claim 2, wherein the radial projection has first and second side faces extending radially from the peripheral surface, and a perimeter face extending from the first side face to the second side face.

5. The introducer sheath assembly of claim 2, wherein the radial projection has an outer surface that extends from the peripheral surface at an oblique angle relative to an axis extending through the proximal and distal faces.

6. The introducer sheath assembly of claim 1, wherein the radial sealing feature comprises a plurality of spaced radial projections each extending radially outward of and circumferentially about the peripheral surface of the main body, wherein each radial projection is in sealing engagement with the inner surface of the housing.

7. The introducer sheath assembly of claim 6, wherein each radial projection has a semi-circular cross-sectional profile.

8. The introducer sheath assembly of claim 6, wherein each radial projection has first and second side faces extending radially from the peripheral surface, and a perimeter face extending from the first side face to the second side face.

9. The introducer sheath assembly of claim 1, wherein the inner surface of the housing includes an annular projection, and wherein the radial sealing feature comprises a concave recess extending radially inward from the peripheral surface, the concave recess having a cross-sectional profile configured to receive the annular projection of the housing in sealing engagement therewith.

10. A hemostasis valve assembly for an introducer sheath having a sheath lumen, the hemostasis valve assembly comprising:
a housing configured to permit introduction of an instrument therethrough, the housing defining an inner surface; and
a seal member configured to inhibit fluid leakage from the hemostasis valve assembly, the seal member comprising:
a main body portion having a proximal face, a distal face, and a peripheral surface extending between the proximal and distal faces, the main body portion having a first diameter defined by the peripheral surface; and
a radial sealing feature extending radially inward or outward from the peripheral surface and configured to sealingly engage the inner surface of the housing.

11. The hemostasis valve assembly of claim 10, wherein the radial sealing feature comprises a radial projection extending radially outward from and circumferentially about the peripheral surface of the main body portion, a radial extremity of the radial projection defining a second diameter greater than the first diameter, wherein the radial projection sealingly engages the inner surface of the housing.

12. The hemostasis valve assembly of claim 11, wherein the radial projection has a semi-circular cross-sectional profile.

13. The hemostasis valve assembly of claim 11, wherein the radial projection has first and second side faces extending radially from the peripheral surface, and a perimeter face extending from the first side face to the second side face.

14. The hemostasis valve assembly of claim 10, wherein the inner surface of the housing includes an annular projection, and wherein the radial sealing feature comprises a concave recess extending radially inward from the peripheral surface, the concave recess having a cross-sectional profile configured to receive the annular projection of the housing in sealing engagement therewith.

15. A hemostasis seal member for a medical device, the seal member comprising:
a generally cylindrical main body portion having a proximal face, a distal face, and a peripheral surface extending between the proximal and distal faces, the main body portion having a first diameter; and
a radial sealing feature extending radially inward or outward from the peripheral surface and configured to sealingly engage an inner surface of a hemostasis valve housing.

16. The hemostasis seal member of claim 15, wherein the radial sealing feature comprises a radial projection extending radially outward from and circumferentially about the peripheral surface of the main body portion, a radial extremity of the radial projection defining a second diameter greater than the first diameter.

17. The hemostasis seal member of claim 16, wherein the radial projection has a semi-circular cross-sectional profile.

18. The hemostasis seal member of claim 16, wherein the radial projection has first and second side faces extending radially from the peripheral surface, and a perimeter face extending from the first side face to the second side face.

19. The hemostasis seal member of claim 15, wherein the radial sealing feature comprises a plurality of spaced radial projections each extending radially outward of and circumferentially about the peripheral surface of the main body.

20. The introducer sheath assembly of claim 15, wherein the radial sealing feature comprises a concave recess extending radially inward from the peripheral surface, the concave recess having a cross-sectional profile configured to receive an annular projection of the housing in sealing engagement therewith.
